(19) United States
(12) Patent Application Publication
(10) Pub. No.: US 2014/0018812 A1
(43) Pub. Date: Jan. 16, 2014

(54) ARTHROPLASTY DEVICE

(71) Applicant: THE CLEVELAND CLINIC FOUNDATION, Cleveland, OH (US)

(72) Inventor: Thomas J. Graham, Novelty, OH (US)

(21) Appl. No.: 13/939,603

(22) Filed: Jul. 11, 2013

Related U.S. Application Data
(60) Provisional application No. 61/670,360, filed on Jul. 11, 2012.

(51) Int. Cl. A61B 17/17

(52) U.S. Cl. CPC A61B 17/1739
USPC 606/87

(57) ABSTRACT

An arthroplasty device for attachment to adjacent first and second bones includes a first member with a base for securing to an extramedullary portion of the first bone and a pin that extends along an axis from the base. A second member has a base for securing to an extramedullary portion of the second bone and a sleeve that extends along an axis from the base and receives the pin of the first member. The pin and sleeve are rotatable relative to one another about the axes when the first and second members are secured to the first and second bones.

### RELATED APPLICATION

This application claims the benefit of U.S. Provisional Appln. No. 61/670,360, filed Jul. 11, 2012, the subject matter of which is incorporated herein in its entirety.

### TECHNICAL FIELD

The invention relates to a surgical device and, in particular, relates to an interphalangeal device that mimics natural articulation between adjacent bones.

### BACKGROUND

The human body includes a myriad of joints that allow various combinations of bones to articulate in order to perform everyday tasks. The joint may deteriorate or lose functionality over time due to numerous factors, e.g., age, trauma, rheumatoid arthritis, osteoarthritis (degenerative joint disease) or psoriatic arthritis. Such deterioration may result in, for example, pain, deformities to the adjoining articulating bones or reduced articulation ability of the bones. It therefore becomes necessary to remove or resect the joint to help prevent further damage to the joint or bones. Many arthroplasty devices, however, require significant resection of the articulating bones while others simply fuse the articulating site. Therefore, there is a need in the art for an arthroplasty device that minimizes bone resection and allows for allows for prescribed articulation between the bones.

### SUMMARY OF THE INVENTION

An arthroplasty device for attachment to adjacent first and second bones in accordance with the present invention includes a first member with a base for securing to an extramedullary portion of the first bone and a pin that extends along an axis from the base. A second member has a base for securing to an extramedullary portion of the second bone and a sleeve that extends along an axis from the base and receives the pin of the first member. The pin and sleeve are rotatable relative to one another about the axes when the first and second members are secured to the first and second bones.

A method for replacing a joint between adjacent first and second bones in accordance with the present invention includes securing a first member having a base and a pin that extends from the base to an extramedullary portion of the first bone. A second member having a base and a sleeve that extends from the base is secured to an extramedullary portion of the second bone such that the pin is received by the sleeve for mimicking natural articulation between the first and second bones.

An arthroplasty device for attachment to adjacent first and second bones in accordance with another aspect of the present invention includes a first member having a base for securing to an extramedullary portion of the first bone and a pin that extends along an axis from the base. A second member has a base for securing to an extramedullary portion of the second bone and a sleeve that extends along an axis from the base and receives the pin of the first member. The pin is received by the sleeve to allow for relative movement between the first and second members only about the axes of the pin and the sleeve.

Other objects and advantages and a fuller understanding of the invention will be had from the following detailed description of the preferred embodiments and the accompanying drawings.

### BRIEF DESCRIPTION OF THE DRAWINGS

FIG. 1 is a schematic illustration of an arthroplasty device secured to first and second bones in accordance with the present invention;

FIG. 2 is a side view of a first member of the arthroplasty device of FIG. 1;

FIG. 3 is a side view of a second member of the arthroplasty device of FIG. 1;

FIG. 4 is an exploded assembly view of the arthroplasty device of FIG. 1;

FIG. 5A is a longitudinal section view of the arthroplasty device of FIG. 1 in an assembled condition;

FIG. 5B is a section view taken along FIG. 5B-5B of a modified version of the arthroplasty device of FIG. 1;

FIG. 6A is a schematic illustration of the first and second bones with the head of the first bone resected;

FIG. 6B is a schematic illustration of the second member secured to the second bone;

FIG. 6C is a schematic illustration of the first and second members connected to one another and secured to the first and second bones;

FIG. 7 is a schematic illustration of an arthroplasty device in accordance with another aspect of the present invention;

FIG. 8 is a sectional view of the arthroplasty device of FIG. 7 secured to the first and second bones;

FIG. 9 is a sectional view of an arthroplasty device in accordance with yet another aspect of the present invention; and

FIG. 10 is a sectional view of an arthroplasty device in accordance with yet another aspect of the present invention.

### DETAILED DESCRIPTION

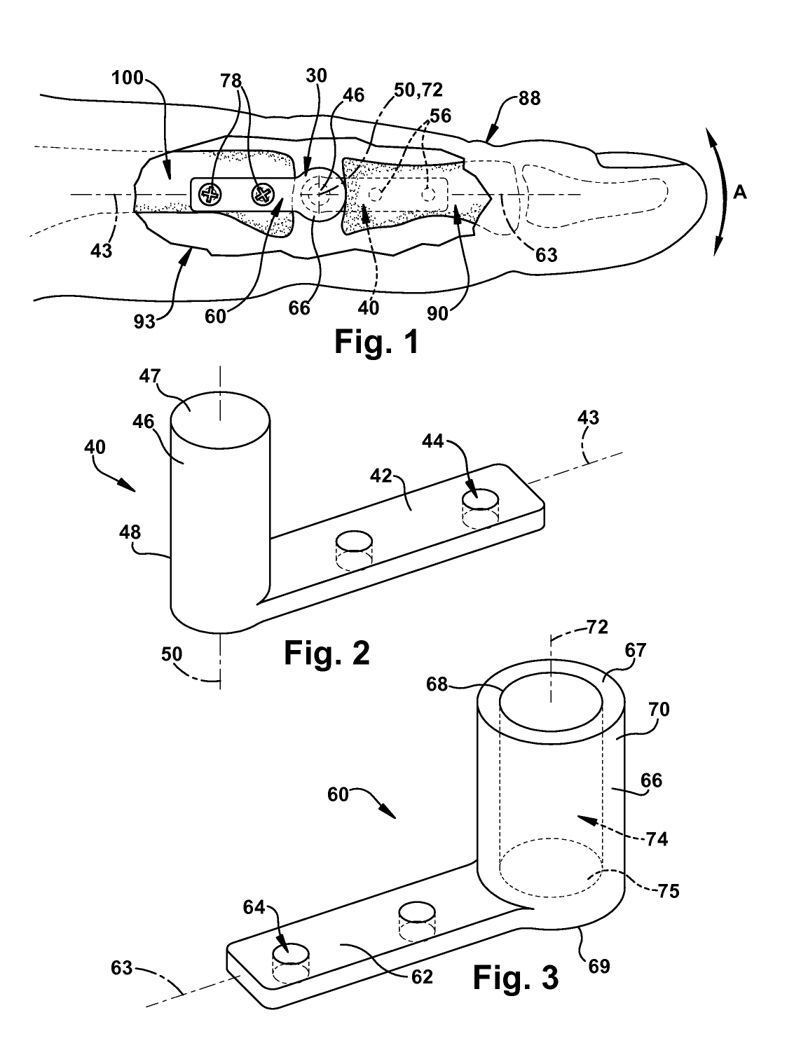

The invention relates to a surgical device and, in particular, relates to an interphalangeal device that mimics natural articulation between adjacent bones. FIGS. 1-3 illustrate an arthroplasty device 30 in accordance with the present invention. The device 30 is implantable between adjacent first and second bones 90, 100 within a mammalian body. As illustrated in FIG. 1, the first bone 90 and second bone 100 constitute the proximal phalanx and metacarpal bone, respectively, in a human finger 88. It will be understood, however, that the device 30 could likewise be implanted between different metacarpal bones, metatarpal bones or any other adjacent, articulating bones in any mammalian body.

The device 30 includes a first member 40 for attachment to the extramedullary portion of the first bone 90 and a second member 60 for attachment to the extramedullary portion of the second bone 100. Referring to FIG. 2, the first member 40 is formed from a biocompatible material and includes a base 42 and a pin 46 that extends from the base. The base 42 is generally planar and extends along an axis 43. One or more openings 44 formed in the base 42 receive fasteners 56 (see FIG. 1) to secure the first member 40 to the first bone 90. The openings 44 may be smooth or threaded (not shown). The base 42 may be mechanically and/or chemically treated to promote fixation of the base to the first bone 90. Although the base 42 is illustrated as being planar, the base may alternatively be contoured, e.g., convex, to the shape of the extramedullary portion of the first bone 90. The pin 46 has an outer surface 48 defining a round or cylindrical shape that extends along an axis 50 and terminates at an end surface 47. The pin 46 extends perpendicular to the base 42 but may likewise extend transversely from the base to meet desired anatomical or performance criterion.

The second member 60 illustrated in FIG. 3 is formed from a biocompatible material and includes a base 62 and a sleeve 66 that extends from the base. The base 62 is generally planar and extends along an axis 63. One or more openings 64 formed in the base 63 receive fasteners 78 (see FIG. 1) to secure the second member 60 to the second bone 100. The openings 64 may be smooth or threaded (not shown). The base 62 of the second member 60 may be mechanically and/or chemically treated to promote fixation of the base to the second bone 100. Although the base 62 is illustrated as being planar the base may alternatively be contoured, e.g., convex, to the shape of the extramedullary portion of the second bone 100.

The sleeve 66 has a round or cylindrical shape that mimics the shape of the pin 46 and extends along an axis 72 at an angle from the base 62. As shown, the sleeve 66 extends perpendicular to the base 62, although the pin may likewise extend transversely from the base to meet desired anatomical or performance criterion. The angle between the sleeve 66 and the base 62 may be the same as or different from the angle between the pin 46 and the base 42.

The sleeve 66 includes an inner surface 68 and an outer surface 70. The inner surface 68 defines a central passage 74 that extends along the axis 72 from an end surface 67 of the sleeve towards the base 62, terminating at an interior surface 75 of the sleeve. The central passage 74 has the same cross-section and substantially the same length along the axis 72 as the pin 46 of the first member 40 has relative to the axis 50. In one example, the inner surface 68 of the sleeve 66 and the outer surface 48 of the pin 46 have concentric, circular cross-sections. Due to this configuration, the central passage 74 of the sleeve 66 is sized to slidably receive the pin 46.

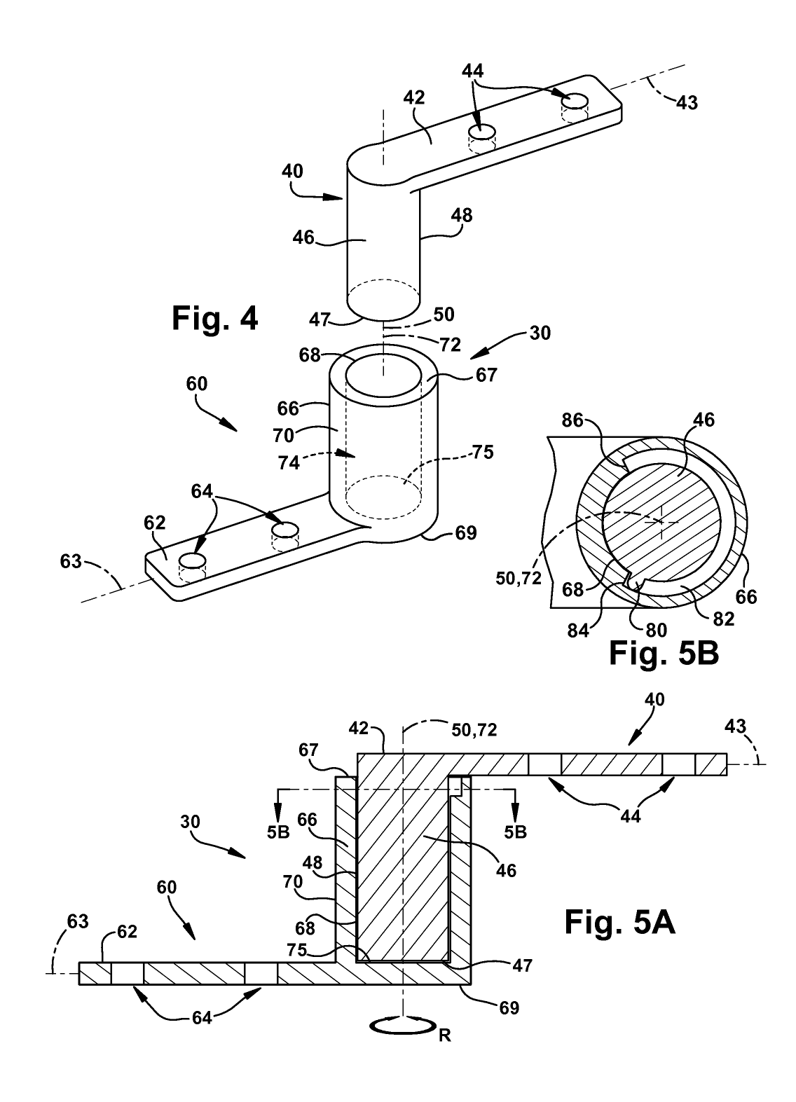

FIGS. 4-6 illustrate assembly of the device 30. As shown in FIG. 4, the axis 50 of the pin 46 is aligned with the axis 72 of the sleeve 66 with the end surface 67 of the sleeve 66 facing the end surface 47 of the pin 46. At this time, the bases 42, 62 of the first and second members 40, 60 may have any angular position relative to the axes 50, 72 and one another, i.e., the bases may extend parallel to one another or may be angled relative to one another. The pin 46 is then inserted into or received by the passage 74 of the sleeve 66 until the base 42 of the first member 40 abuts the end surface 67 of the sleeve of the second member 60 (FIG. 5A). Alternatively or additionally, the pin 46 is inserted into the passage 74 until the end surface 47 abuts the interior surface 75 of the sleeve 66. In each case, the pin 46 and sleeve 66 are free to rotate relative to one another about the axes 50, 72 as indicated generally by the arrow “R” in FIG. 5A.

Referring to FIG. 5B, the sleeve 66 and the pin 46 may include cooperating structure that limits relative rotation between the first and second members 40, 60 in the direction R to a prescribed degree of freedom about the axes 50, 72 to meet particular anatomical conditions and/or performance criterion. In one example, the pin 46 includes a radially extending projection 80 that cooperates with an arcuate recess 82 formed in the inner surface 68 of the sleeve 66. The arcuate recess 82 extends circumferentially about the axis 70 between a pair of end stops 84, 86 that define the angular path over which the pin 46 and sleeve 66 may rotate relative to one another in the direction R. The angular path of movement prescribed between the end stops 84, 86 is comparable to the natural range of angular motion between the first and second bones 90, 100. In one example, the end stops 84, 86 define an arc exceeding 180°, although the end stops may be spaced closer together.

A sleeve, lining or lubricating coating (not shown) may be provided along the inner surface 68 of the second member 60 and/or along the outer surface 48 of the pin 46 for reducing wear between the first and second members 40, 60 when the first and second members rotate relative to one another. The sleeve or lining may be removably secured to the inner surface 68 of the sleeve 66 and/or the outer surface 48 of the pin 46 to allow for replacement over time. The material used for the sleeve, lining or coating may constitute a biocompatible, wear-resistant polymer, e.g., polyether ether ketones (PEEK), polycarbons, and copolymers and blends thereof. Known lubricating fluids may also be used in accordance with the present invention.

The inner surface 68 of the sleeve 66 and the outer surface 48 of the pin 46 are sized to substantially or entirely prevent movement of the pin relative to the sleeve in directions other than about the axes 50, 72 once the device 30 is implanted. In other words, the pin 46 and sleeve 66 are sized and shaped such that relative movement between the pin and sleeve is substantially limited to rotation about the axes 50, 72. Therefore, the pin 46 and sleeve 66 do not tilt relative to one another about the axes 50, 72 when the device 30 is implanted. Additionally, the pin 46 and sleeve 66 do not move longitudinally relative to one another along the axes 43, 63 or rotate relative to one another about the axes 43, 63. To this end, the diameter of the passage 74 and the diameter of the pin 46 are substantially similar to one another to form the appropriate fit between the first and second members 40, 60, e.g., sliding fit that approaches a press or interference fit.

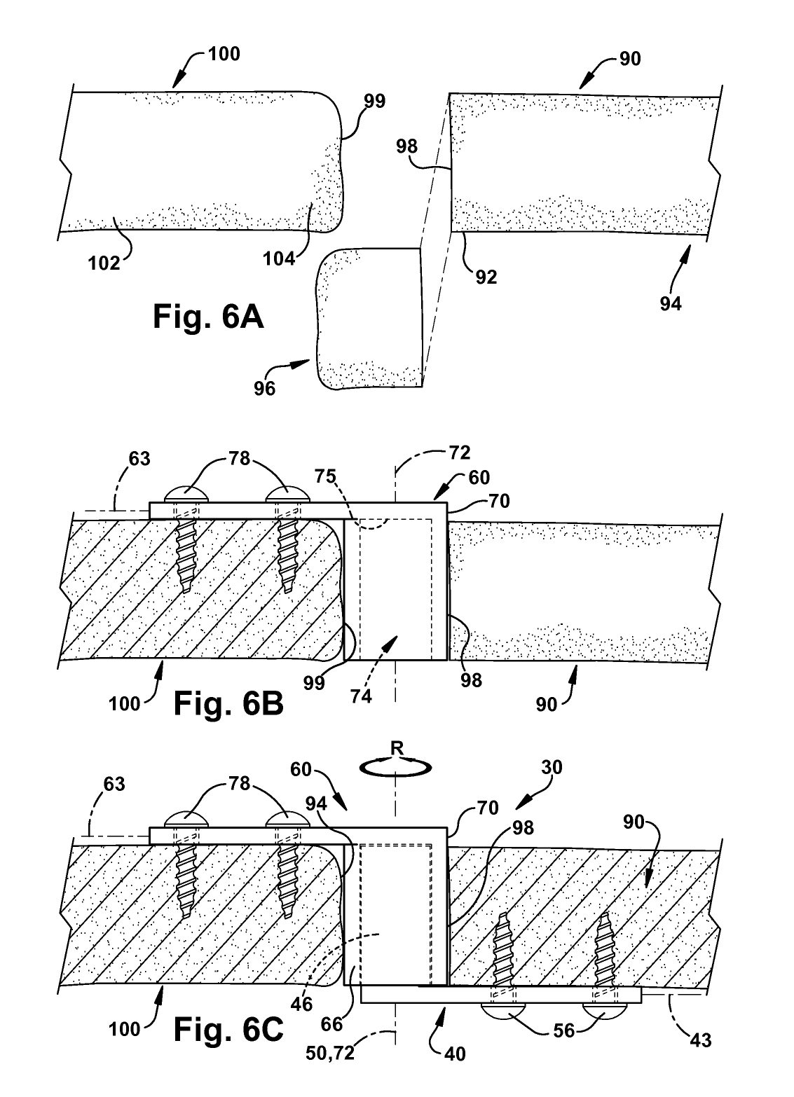

FIGS. 6A-C illustrate steps for implanting the device 30 between first and second metacarpal bones 90, 100 in accordance with the present invention. The first bone 90 may be proximal to the second bone 100 or may be distal to the second bone. In any case, a desired implantation site between the bones 90, 100 is accessed with a small incision 93 made along the finger 88 (see FIG. 1) adjacent to the joint to be removed. The incision 93 and subsequent implantation of the device 30 is performed with the finger 88 articulated to a position between full extension and full flexion (not shown). The bones 90, 100 are prepared prior to receiving the device 30 therebetween by excising the head 96 of the first bone 90 to expose an end surface 98 of the first bone (FIG. 6A). The end surface 98 can be shaped to the contour of the outer surface 70 of the sleeve 66 of the second member 60. An end surface 99 of the second bone 100 facing the end surface 98 of the first bone 90 may also be shaped to the contour of the outer surface 70 of the sleeve 66.

Referring to FIG. 6B, the second member 60 is secured to the second bone 100. In particular, the sleeve 66 is positioned between the first and second bones 90, 100 and the base 62 of the second member 60 is positioned on the top side of the second bone 100 (as viewed in FIG. 6B). It will be understood, however, that the base 62 of the second member 60 could be positioned along any side or surface, e.g., bottom or lateral surface, of the second bone 100 so long as the sleeve 66 is positioned between the first and second bones 90, 100. In any case, the axis 63 of the base 62 extends generally parallel to the length of the second bone 100. In this condition, the outer surface 70 of the sleeve 66 may engage one, both or neither end surface 98, 99 of the first and second bones 90, 100, respectively. Fasteners 78 are inserted or threaded through the openings 64 in the base 62 in the second member 60 and threaded into the second bone 100 to secure the second member to the second bone. Although threaded fasteners 78 are illustrated, it will be appreciated that the second member 60 could be secured to the second bone 100 using any fastening means, e.g., mechanical, chemical, and/or adhesive, etc. Regardless, once the sleeve 66 is secured to the second bone 100 the axis 72 of the sleeve extends substantially perpendicular to the direction of extension of the first and second bones 90, 100. In other words, the axis 72 extends perpendicular to the general plane of the first and second bones 90, 100 with any degree of flexion or extension in the finger 88.

Referring to FIG. 6C, the first member 40 is secured to the first bone 90 once the second member 60 is secured to the second bone 100. To this end, the pin 46 is inserted into or received by the sleeve 66 and the base 42 of the first member 40 is positioned on the bottom side of the first bone 90 (as viewed in FIG. 6C). It will be understood, however, that the base 42 of the first member 40 could be positioned along any side or surface of the first bone 90, e.g., bottom or lateral surface, so long as the pin 46 is positioned within the sleeve 66 of the second member 60. In this configuration, the base 42 of the first member 40 and the base 62 of the second member 60 are positioned on opposite sides of the first and second bones 90, 100, respectively, from one another. Fasteners 56 are inserted or threaded through the openings 44 in the base 42 of the first member 40 and threaded into the first bone 90 to secure the first member to the first bone. Although threaded fasteners 56 are illustrated, it will be appreciated that the first member 56 could be secured to the first bone 90 using any fastening means, e.g., mechanical, chemical, and/or adhesive, etc.

Although FIGS. 6A-C illustrate that the second member 60 is secured to the second bone 100 prior to securing the first member 40 to the first bone 90, it will be appreciated that the first member 40 can be secured to the second bone 100 and the second member 60 can be secured to the first bone 90 in accordance with the present invention. In any case, once both members 40, 60 are secured to the respective bone 90, 100 the axis 50 of the pin 46 extends substantially perpendicular to the direction of extension of the first and second bones. The axis 50 of the pin 46 is therefore coincident with the axis 72 of the sleeve 66. Consequently, the device 30 is secured to the first and second bones 90, 100 such that that axes 50, 72 are aligned with the natural axis of rotation of the resected joint between the first and second bones. Due to this configuration, the device 30 allows for relative movement between the first and second bones 90, 100 in a natural manner.

The device 30 of the present invention therefore mimics natural articulation between the first and second bones 90, 100 by replacing the resected joint. To this end, due to the natural geometry of the first and second bones 90, 100 and the positioning of the device 30, the first and second members 40, 60 are prevented from moving relative to one another along the axes 50, 72 or about the axes 43, 63 while being permitted to rotate relative to one another about the axes 50, 72.

Referring to FIG. 1, relative movement between the first and second bones 90, 100 is facilitated and controlled by the device 30 once the device is implanted between the first and second bones. When either the first or second bone 90 or 100 moves the member 40 or 60 secured thereto via the respective base 42, 62 also moves. The construction of the device 30 allows the bones 90, 100 to pivot, i.e., articulate relative to one another about the axes 50, 72, as illustrated generally by the arrow A in FIG. 1. For the reasons discussed, however, the bones 90, 100 are limited or prevented from sliding or shifting relative to one another along the axes 50, 72 or tilting relative to one another about the axes 43, 63. By restricting the allowable relative movement between the bones 90, 100, the device 30 of the present invention advantageously mimics natural articulation of the finger 88 at the joint while preventing unnatural or unwanted movement between the bones that could result in damage to the surrounding tissue, tendons, etc.

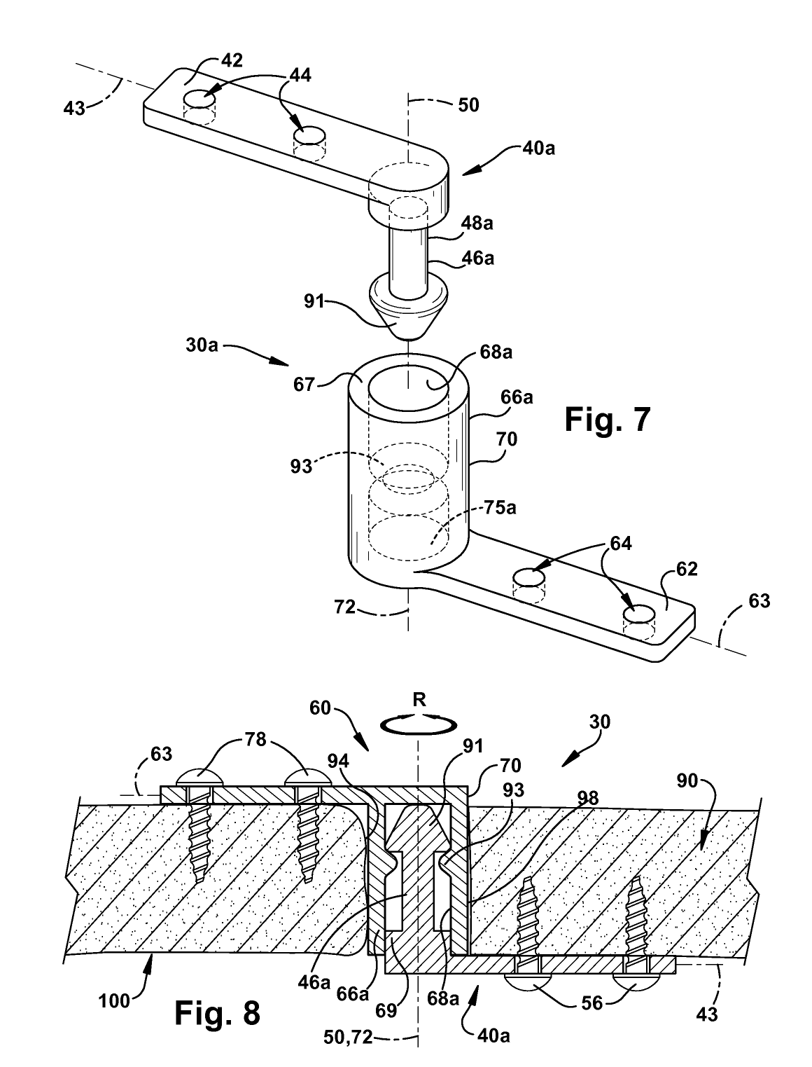

An arthroplasty device 30a in accordance with another aspect of the present invention is illustrated in FIGS. 7-8. Features in FIGS. 7-8 that are similar to those illustrated in FIGS. 1-6C are given the same reference number whereas features in FIGS. 7-8 that are different than those illustrated in FIGS. 1-6C are given the suffix “a”. In FIGS. 7-8, the pin 46a and the sleeve 66a form a snap-fit connection with one another. Referring to FIG. 7, the pin 46a includes a cooperating member 91 at its terminal end and the sleeve 66a includes a corresponding cooperating member 93 along the passage 74. The cooperating members 91, 93 may be formed from a resilient or flexible material that elastically deforms under the application of force.

The cooperating member 91 of the pin 46a constitutes an enlarged or flanged portion that projects radially outward from the axis 50 further than the remainder of the pin. In one example, the cooperating member 91 has a tapered or substantially conical shape, although other shapes are contemplated. The cooperating member 93 is formed on the inner surface 68a of the sleeve 66a near the base 62. The cooperating member 93 may constitute a single, annular flange or a series of projections centered about the axis 72 of the sleeve (not shown) and extending radially inward from the inner surface 68a towards the axis 72.

As with the device 30a, the joint is accessed and resected and the first and second members 40a, 60a are secured via fasteners 56, 72 to the respective first and second bones 90, 100. Once one of the first and second members 40a or 60a is in place, the remaining member is secured to the corresponding bone 90 or 100 while aligning the axes 50, 72 and securing the cooperating members 91, 93 together in a snap-fit fashion.

When the device 30a is secured to the bones 90, 100 (see FIG. 8) the cooperating members 91, 93 connect to one another to allow for relative rotation between the first and second members 40a, 60a about the axes 50, 72 while preventing or substantially limiting relative movement along the axes 50, 72 and about the axes 43, 63. More specifically, the cooperating members 91, 93 may be formed from a resilient or flexible material that elastically deforms to allow the enlarged portion 91 of the pin 46a is inserted into or received by the passage 74a of the sleeve 66a, elastically deforms to pass the flange 93, and ultimately bottoms out on the surface 75a of the sleeve. This wedges the cooperating member 91 between the cooperating member 93 and the surface 75a to prevent relative movement between the pin 46a and the sleeve 66a along the axes 50, 72 and about the axes 43, 63. The cooperating member 91 may, however, rotate between and relative to the bottom surface 75a and the cooperating member 93 about the axes 50, 72.

The pin 46a may include an enlarged portion 69 near the base 62 that is sized to fit within and slide along the inner surface 68a of the sleeve 66a. Such a configuration helps to maintain the pin 46a and sleeve 66a in a coaxial relationship during operation of the device 30a, thereby promoting smooth relative rotation between the first and second members 40a, 60a.

Although FIGS. 7-8 illustrates snap-fit cooperating members 91, 93 it will be appreciated that the pin 46a and sleeve 66a of the present invention may include alternative mechanical configurations that cooperate with one another in order to releasably connect the first and second members 40a, 60a together in a manner that allows for relative rotation between the pin 46a and sleeve 66a about the axes 50, 72 but prevents relative longitudinal and tilting movement along the axes 43, 63.

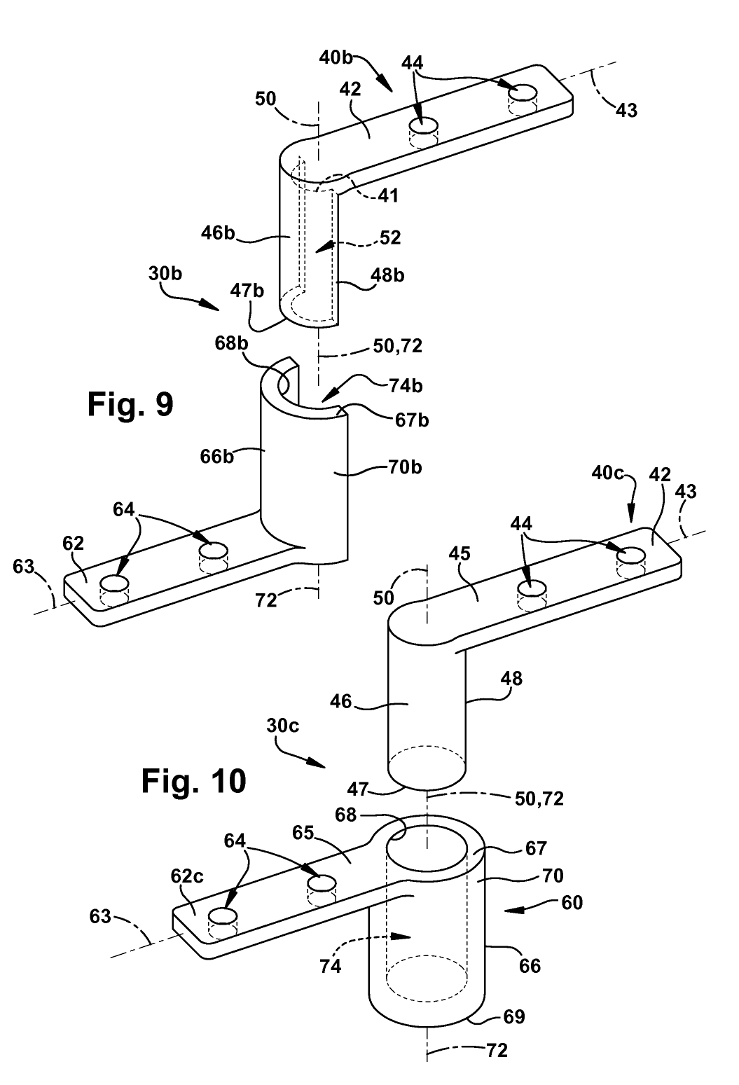

An arthroplasty device 30b in accordance with another aspect of the present invention is illustrated in FIG. 9. Features in FIG. 9 that are similar to those illustrated in FIGS. 1-6C are given the same reference number whereas features in FIG. 9 that are different than those illustrated in FIGS. 1-6C are given the suffix “b”. In FIG. 9, the sleeve 66b and the pin 46b have different configurations than the sleeve 66 and the pin 46, respectively. In particular, the pin 46b has an arcuate or semi-circular cross-section that extends circumferentially about the axis 50. Although the pin 46b is shown extending roughly 180° about the axis 50, it will be appreciated that the pin may extend about the axis by a greater or lesser degree.

The pin 46b includes an inner surface 41 and an outer surface 48b. The inner and outer surfaces 41, 48b may be concentric with one another or may be different from one another. The inner surface 41 may be shaped to mimic the contour of the end surface 98 of the first bone 90 (see FIG. 6A). The inner surface 41 defines a blind passage 52 that extends along the axis 50 from the end surface 47b of the pin 46b to the base 42. The passage 52 accommodates a portion of the first bone 90 when the first member 40b is secured to the first bone (not shown). The passage 52 is laterally accessible by the first bone 90 such that the first bone can be received by the passage in a direction other than along the axis 72.

The sleeve 66b has an arcuate or semi-circular cross-section that extends circumferentially about the axis 72. Although the sleeve 66b shown as extending roughly 180° about the axis 72, it will be appreciated that the sleeve may extend about the axis a greater or lesser degree. The sleeve 66b includes an inner surface 68b and an outer surface 70b. The inner and outer surfaces 68b, 70b may be concentric with one another or may be different from one another. The inner surface 68b is shaped to the contour of the outer surface 48b of the pin 46b and cooperates therewith to allow for smooth relative rotation between the first member 40b and the second member 60b during operation of the device 30. The outer surface 70b may be shaped to the contour of the end surface 99 of the second bone 100 (see FIG. 6A). The inner surface 68b defines the passage 74b, which extends along the axis 72 and entirely through the sleeve 66b. The passage 74b accommodates the pin 46b when the first member 40b is secured to the first bone 90 and the second member 60b is secured to the second bone 100 (not shown). The passage 74b is laterally accessible by the second bone 100 such that the second bone can be received by the passage in a direction other than along the axis 50.

The device 30b is assembled and secured to the first and second bones 90, 100 in a manner similar to that described in FIGS. 6A-C. In the device 30b, however, the passage 74b permits lateral insertion of the pin 46b into the sleeve 66b. Therefore, the user can assemble the device 30b by either aligning the axes 50, 72 while inserting the pin 46b into the sleeve 66a or by inserting the pin laterally through the passage 74b into the sleeve. In either case, the sleeve 66b receives and engages the pin 46b to allow for relative rotation therebetween about the axes 50, 70. The configuration of the first and second members 40b, 60b, as well as the geometry of the end surfaces 98, 99 of the first and second bones 90, 100, ensures that the pin 48b remains engaged with the inner surface 68b of the sleeve 66b during use of the device 30b. Moreover, the pin 46b and sleeve 66b are prevented from moving longitudinally along the axes 50, 72 or moving/tilting relative to the axes 43, 63 or one another during use. Accordingly, the device 30b only allows for relative rotation between the first and second members 40b, 60b about the axes 50, 70 during use.

An arthroplasty device 30c in accordance with another aspect of the present invention is illustrated in FIG. 10. Features in FIG. 10 that are similar to those illustrated in FIGS. 1-6C are given the same reference number whereas features in FIG. 10 that are different than those illustrated in FIGS. 1-6C are given the suffix “c”. In FIG. 10, the first member 40c and second member 60a are configured to be secured unilaterally to the first and second bones 90, 100, respectively. In other words, the first and second members 40c, 60c are secured to the same side of the respective first and second bones 90, 100, e.g., top side, bottom side or a lateral side.

To this end, the base 62c of the second member 60c is located adjacent to the end surface 67 of the sleeve 66 such that the end surface 63 of the base is substantially co-planar with the end surface of the sleeve when the device 30a is implanted. When the pin 46 is fully inserted into or received by the passage 74 of the sleeve 66, a surface 45 of the base 42 and a surface 65 of the sleeve 66 are substantially co-planar with one another or have substantially similar longitudinal positions along the axes 50, 72. Due to this configuration, the base 42 of the first member 40c and the base 62c of the second member 60c can be positioned on and secured to the same side of the first bone 90 and the second bone 100.

The arthroplasty device of the present invention is advantageous for several reasons. The connection between the first and second members, namely, between the pin and the sleeve, provides stability to the device and restricts relative movement between the bones in order to help prevent injury and reduce wear on the components. The arthroplasty device may therefore limit or prevent flexion and extension manipulations. The compact design of the device also minimizes dissection of the head(s) of the bones and facilitates salvage (fusion).

The preferred embodiments of the invention have been illustrated and described in detail. However, the present invention is not to be considered limited to the precise construction disclosed. Various adaptations, modifications and uses of the invention may occur to those skilled in the art to which the invention relates and the intention is to cover hereby all such adaptations, modifications, and uses which fall within the spirit or scope of the appended claims.

Having described the invention, the following is claimed:

1. An arthroplasty device for attachment to adjacent first and second bones comprising:
a first member having a base for securing to an extramedullary portion of the first bone and a pin extending along an axis from the base; and
a second member having a base for securing to an extramedullary portion of the second bone and a sleeve extending along an axis from the base and receiving the pin of the first member, the pin and sleeve being rotatable relative to one another about the axes when the first and second members are secured to the first and second bones.

2. The arthroplasty device of claim 1, wherein the base of the first member includes at least one opening for receiving a fastener to secure the first member to the extramedullary portion of the first bone.

3. The arthroplasty device of claim 2, wherein the base of the second member includes at least one opening for receiving a fastener to secure the second member to the extramedullary portion of the second bone.

4. The arthroplasty device of claim 1, wherein the first member and the second member are secured to the same side of the first bone and the second bone.

5. The arthroplasty device of claim 1, wherein the first bone and the second bone are one of metacarpal bones and metatarsal bones.

6. The arthroplasty device of claim 1, wherein the pin and the sleeve cooperate to limit relative movement between the first member and the second member to relative rotation about the axes of the pin and the sleeve.

7. The arthroplasty device of claim 1, wherein the pin includes a portion that releasably engages a portion of the sleeve to allow for relative rotation between the first and second members about the axes of the pin and the sleeve but prevents relative longitudinal movement between the first and second members along the axes.

8. The arthroplasty device of claim 7, wherein an enlarged portion extends radially outward from the pin and an annular flange extends radially inward from the sleeve, the enlarged portion and annular flange forming a snap-fit connection that prevents relative movement between the first and second members along the axes of the pin and the sleeve.

9. The arthroplasty device of claim 1, wherein the pin includes a lateral passage for receiving a portion of the first bone.

10. The arthroplasty device of claim 1, wherein the pin has an arcuate cross-section extending partially about the axis of the pin and the sleeve has an arcuate cross-section extending partially about the axis of the sleeve.

11. The arthroplasty device of claim 1, wherein the base of the pin extends along an axis of the base of the sleeve extends along an axis, the pin and the sleeve being prevented from moving longitudinally along the axes of the bases of the pin and the sleeve and tilting about the axes of the bases of the pin and the sieve.

12. A method for replacing a joint between adjacent first and second bones comprising:
securing a first member having a base and a pin extending from the base to an extramedullary portion of the first bone; and
securing a second member having a base and a sleeve extending from the base to an extramedullary portion of the second bone such that the pin is received by the sleeve for mimicking natural articulation between the first and second bones.

13. The method of claim 12, wherein the sleeve is connected to the pin such that relative movement between the first member and the second member is restricted to only relative movement about an axis extending through the pin of the first member.

14. The method of claim 12, wherein securing the first and second members to the first and second bones comprises securing the first and second members to the same side of the first bone and the second bone.

15. The method of claim 12 further comprising laterally inserting the pin into the sleeve until the pin and sleeve are coaxial with one another.

16. An arthroplasty device for attachment to adjacent first and second bones comprising:
a first member having a base for securing to an extramedullary portion of the first bone and a pin extending along an axis from the base; and
a second member having a base for securing to an extramedullary portion of the second bone and a sleeve extending along an axis from the base and receiving the pin of the first member, the pin being received by the sleeve to allow for relative movement between the first and second members only about the axes of the pin and the sleeve.

17. The arthroplasty device of claim 16, wherein the pin includes a portion that releasably engages a portion of the sleeve to allow for relative rotation between the first and second members about the axes of the pin and the sleeve but prevents relative longitudinal movement between the first and second members along the axes.

18. The arthroplasty device of claim 17, wherein an enlarged portion extends radially outward from the pin and an annular flange extends radially inward from the sleeve, the enlarged portion and annular flange forming a snap-fit connection that prevents relative movement between the first and second members along the axes of the pin and the sleeve.

19. The arthroplasty device of claim 16, wherein the pin includes a lateral passage for receiving a portion of the first bone.

20. The arthroplasty device of claim 16, wherein the pin has an arcuate cross-section extending partially about the axis of the pin and the sleeve has an arcuate cross-section extending partially about the axis of the sleeve.
